(19) United States
(12) Patent Application Publication
(10) Pub. No.: US 2025/0146994 A1
(43) Pub. Date: May 8, 2025

(54) APPARATUS AND METHOD FOR CONTROLLING SPATIALLY SELECTIVE AIR SAMPLING

(71) Applicant: ELECTRONICS AND TELECOMMUNICATIONS RESEARCH INSTITUTE, Daejeon (KR)

(72) Inventors: You Jin KIM, Daejeon (KR); Kwang Hyo CHUNG, Daejeon (KR); Dae Won KIM, Daejeon (KR); Jae Young JUNG, Daejeon (KR)

(21) Appl. No.: 18/752,934

(22) Filed: Jun. 25, 2024

(30) Foreign Application Priority Data
Nov. 7, 2023 (KR) 10-2023-0153016

(51) Int. Cl. G01N 33/00

(52) U.S. Cl. CPC G01N 33/0075; G01N 33/0034; G01N 33/0068

(57) ABSTRACT

Provided are an apparatus and a method of controlling spatially selective air sampling, the apparatus including a sensing unit configured to detect a multimodal sensing value for at least one space, a diagnostic module configured to collect fine suspended materials in air of the space and diagnose composition of the fine suspended materials, an air adjuster configured to suction the air of the space and transfer the suctioned air to the diagnostic module, and a processor configured to generate color palette data of a unified data space having pixel brightness corresponding to signal intensity of the multimodal sensing value, map the color palette data to a diagnostic result of the diagnostic module, perform inference through a learning model based on the color palette data and the diagnostic result, and control the air adjuster for each space according to an inference result to transfer the air to the diagnostic module.

### CROSS-REFERENCE TO RELATED APPLICATION

This application claims priority to and the benefit of Korean Patent Application No. 10-2023-0153016, filed on Nov. 7, 2023, the disclosure of which is incorporated herein by reference in its entirety.

### BACKGROUND

### 1. Field of the Invention

The present invention relates to an apparatus and a method for controlling spatially selective air sampling.

### 2. Description of Related Art

When objects such as people or animals live in a specific crowded space, in the case in which an environment (for example, temperature, humidity, or CO₂) of the space is detected and an interaction between the objects (for example, an image, a voice, or a noise magnitude) is detected, there is a problem of having to manage data individually due to the unimodality characteristics resulting from different sensing data spaces.

For example, data detected through an image is managed as image data, and environmental sensing data such as temperature, humidity, or CO₂ is managed as numeric data. However, in terms of a data space, image data and environmental data are present in independent data spaces, and thus there is no intersecting data space.

Meanwhile, in order to map sensing data, which consists of multimodality which is a bundle of individual and allogeneous unimodalities, to one complex unified data space, when contextual translation is required, there is a problem that a complex process is required.

In addition, in the case of air sampling devices for diagnosing the characteristics of fine suspended materials in air, at least one diagnostic device is generally installed in one space to perform a diagnosis. In the case of a plurality of spaces, there is a problem that a plurality of air sampling devices are required to be installed in each space.

The background technology of the present invention is disclosed in Korean Patent Publication No. 10-2023-0117950 (published on Aug. 10, 2023).

### SUMMARY OF THE INVENTION

The present invention is directed to providing an apparatus and a method for controlling spatially selective air sampling, which maps a multimodal sensing value with multimodality characteristics such as image data, voice data, an analog signal, and a digital signal to one unified data space.

According to an aspect of the present invention, there is provided an apparatus for controlling spatially selective air sampling, the apparatus including a sensing unit configured to detect a multimodal sensing value for at least one space, a diagnostic module configured to collect fine suspended materials in air of the space and diagnose a composition of the fine suspended materials, an air adjuster configured to suction the air of the space and transfer the suctioned air to the diagnostic module, and a processor configured to generate color palette data of a unified data space having pixel brightness corresponding to signal intensity of the multimodal sensing value, map the color palette data to a diagnostic result of the diagnostic module, perform inference through a learning model based on the color palette data and the diagnostic result, and control the air adjuster for each space according to an inference result to transfer the air to the diagnostic module.

The diagnostic module may include a collection unit configured to collect the fine suspended materials in the air of the air adjuster, and a diagnostic unit configured to diagnose the composition of the fine suspended materials in the collection unit.

The air adjuster may include a duct connected to the space to transfer the air suctioned from the space through an air tube to the diagnostic module, and an internal air valve installed on the air tube to block the air in the space and introduce the air into the duct.

The apparatus may further include a filter module configured to suction and filter outside air, and supply the filtered outside air to the duct.

The processor may define a rate of a sensing range of the multimodal sensing value, may map the sensing range to a color palette, and may generate individual color collections for each preset setting cycle.

The processor may generate the color palette data by combining the individual color collections in a preset time section.

The apparatus may further include a model generator configured to decode the individual color collection to obtain an individual situation, generate a contextual definition result according to time information with the individual situation, convert the contextual definition result into a meta-data label according to contextual translation of a user, match the meta-data label with the color palette data to constitute a dataset, and then train the learning model using the dataset.

According to another aspect of the present invention, there is provided an apparatus for controlling spatially selective air sampling, the apparatus including a processor, and a memory configured to store an instruction executed by the processor, wherein the processor uses a multimodal sensing value for at least one space to generate color palette data of a unified data space having pixel brightness corresponding to signal intensity of the multimodal sensing value.

The processor may define a rate of the multimodal sensing value to a sensing range, may map the sensing range to a color palette, and may generate individual color collections for each preset setting cycle.

The processor may generate the color palette data by combining the individual color collections in a preset time section.

The learning model may decode the individual color collection to obtain an individual situation, may generate a contextual definition result according to time information with the individual situation, may convert the contextual definition result into a meta-data label according to a contextual translation of a user, may match the meta-data label with the color palette data to constitute a dataset, and then may be trained using the dataset

According to still another aspect of the present invention, there is provided a method of controlling spatially selective air sampling, the method including detecting, by a sensing g unit, a multimodal sensing value for at least one space, collecting, by a diagnostic module, fine suspended materials in air of the space and diagnosing a composition of the fine suspended materials, generating, by a processor, color palette data in a unified data space having pixel brightness corresponding to signal intensity of the multimodal sensing value using the multimodal sensing value, mapping, by the processor, the color palette data to a diagnostic result of the diagnosis module, and performing, by a processor, inference, based on the color palette data and the diagnostic result using a learning model, controlling an air adjuster for each space according to an inference result, and transferring the air to the diagnosis module.

In the generating of the color palette data, the processor may define a rate of a sensing range of the multimodal sensing value, may map the sensing range to a color palette, and may generate individual color collections for each preset setting cycle.

In the generating of the color palette data, the processor may generate the color palette data by combining the individual color collections in a preset time section.

The learning model may decode the individual color collection to obtain an individual situation, may generate a contextual definition result according to time information with the individual situation, may convert the contextual definition result into a meta-data label according to a contextual translation of a user, may match the meta-data label with the color palette data to constitute a dataset, and then may be trained using the dataset.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram of an apparatus for controlling spatially selective air sampling according to one embodiment of the present invention;

FIG. 2 is a block diagram of a processor according to one embodiment of the present invention;

FIG. 3 is a block diagram of a color palette generator (210) according to one embodiment of the present invention;

FIG. 4 is a diagram illustrating a model generating process of a model generator according to one embodiment of the present invention; and

FIG. 5 is a flowchart of a method of controlling spatially selective air sampling according to one embodiment of the present invention.

### DETAILED DESCRIPTION OF EXEMPLARY EMBODIMENTS

The components described in the example embodiments may be implemented by hardware components including, for example, at least one digital signal processor (DSP), a processor, a controller, an application-specific integrated circuit (ASIC), a programmable logic element, such as an FPGA, other electronic devices, or combinations thereof. At least some of the functions or the processes described in the example embodiments may be implemented by software, and the software may be recorded on a recording medium. The components, the functions, and the processes described in the example embodiments may be implemented by a combination of hardware and software.

The method according to example embodiments may be embodied as a program that is executable by a computer, and may be implemented as various recording media such as a magnetic storage medium, an optical reading medium, and a digital storage medium.

Various techniques described herein may be implemented as digital electronic circuitry, or as computer hardware, firmware, software, or combinations thereof. The techniques may be implemented as a computer program product, i.e., a computer program tangibly embodied in an information carrier, e.g., in a machine-readable storage device (for example, a computer-readable medium) or in a propagated signal for processing by, or to control an operation of a data processing apparatus, e.g., a programmable processor, a computer, or multiple computers. A computer program(s) may be written in any form of a programming language, including compiled or interpreted languages and may be deployed in any form including a stand-alone program or a module, a component, a subroutine, or other units suitable for use in a computing environment. A computer program may be deployed to be executed on one computer or on multiple computers at one site or distributed across multiple sites and interconnected by a communication network.

Processors suitable for execution of a computer program include, by way of example, both general and special purpose microprocessors, and any one or more processors of any kind of digital computer. Generally, a processor will receive instructions and data from a read-only memory or a random access memory or both. Elements of a computer may include at least one processor to execute instructions and one or more memory devices to store instructions and data. Generally, a computer will also include or be coupled to receive data from, transfer data to, or perform both on one or more mass storage devices to store data, e.g., magnetic, magneto-optical disks, or optical disks. Examples of information carriers suitable for embodying computer program instructions and data include semiconductor memory devices, for example, magnetic media such as a hard disk, a floppy disk, and a magnetic tape, optical media such as a compact disk read only memory (CD-ROM), a digital video disk (DVD), etc. and magneto-optical media such as a floptical disk, and a read only memory (ROM), a random access memory (RAM), a flash memory, an erasable programmable ROM (EPROM), and an electrically erasable programmable ROM (EEPROM) and any other known computer readable medium. A processor and a memory may be supplemented by, or integrated into, a special purpose logic circuit.

The processor may run an operating system (OS) and one or more software applications that run on the OS. The processor device also may access, store, manipulate, process, and create data in response to execution of the software. For purpose of simplicity, the description of a processor device is used as singular; however, one skilled in the art will be appreciated that a processor device may include multiple processing elements and/or multiple types of processing elements. For example, a processor device may include multiple processors or a processor and a controller. In addition, different processing configurations are possible, such as parallel processors.

Also, non-transitory computer-readable media may be any available media that may be accessed by a computer, and may include both computer storage media and transmission media.

The present specification includes details of a number of specific implements, but it should be understood that the details do not limit any invention or what is claimable in the specification but rather describe features of the specific example embodiment. Features described in the specification in the context of individual example embodiments may be implemented as a combination in a single example embodiment. In contrast, various features described in the specification in the context of a single example embodiment may be implemented in multiple example embodiments individually or in an appropriate sub-combination. Furthermore, the features may operate in a specific combination and may be initially described as claimed in the combination, but one or more features may be excluded from the claimed combination in some cases, and the claimed combination may be changed into a sub-combination or a modification of a sub-combination.

Similarly, even though operations are described in a specific order on the drawings, it should not be understood as the operations needing to be performed in the specific order or in sequence to obtain desired results or as all the operations needing to be performed. In a specific case, multitasking and parallel processing may be advantageous. In addition, it should not be understood as requiring a separation of various apparatus components in the above described example embodiments in all example embodiments, and it should be understood that the above-described program components and apparatuses may be incorporated into a single software product or may be packaged in multiple software products.

It should be understood that the example embodiments disclosed herein are merely illustrative and are not intended to limit the scope of the invention. It will be apparent to one of ordinary skill in the art that various modifications of the example embodiments may be made without departing from the spirit and scope of the claims and their equivalents.

Hereinafter, with reference to the accompanying drawings, embodiments of the present disclosure will be described in detail so that a person skilled in the art can readily carry out the present disclosure. However, the present disclosure may be embodied in many different forms and is not limited to the embodiments described herein.

In the following description of the embodiments of the present disclosure, a detailed description of known functions and configurations incorporated herein will be omitted when it may make the subject matter of the present disclosure rather unclear. Parts not related to the description of the present disclosure in the drawings are omitted, and like parts are denoted by similar reference numerals.

In the present disclosure, components that are distinguished from each other are intended to clearly illustrate each feature. However, it does not necessarily mean that the components are separate. That is, a plurality of components may be integrated into one hardware or software unit, or a single component may be distributed into a plurality of hardware or software units. Thus, unless otherwise noted, such integrated or distributed embodiments are also included within the scope of the present disclosure.

In the present disclosure, components described in the various embodiments are not necessarily essential components, and some may be optional components. Accordingly, embodiments consisting of a subset of the components described in one embodiment are also included within the scope of the present disclosure. In addition, embodiments that include other components in addition to the components described in the various embodiments are also included in the scope of the present disclosure.

Hereinafter, with reference to the accompanying drawings, embodiments of the present disclosure will be described in detail so that a person skilled in the art can readily carry out the present disclosure. However, the present disclosure may be embodied in many different forms and is not limited to the embodiments described herein.

In the following description of the embodiments of the present disclosure, a detailed description of known functions and configurations incorporated herein will be omitted when it may make the subject matter of the present disclosure rather unclear. Parts not related to the description of the present disclosure in the drawings are omitted, and like parts are denoted by similar reference numerals.

In the present disclosure, when a component is referred to as being “linked,” “coupled,” or “connected” to another component, it is understood that not only a direct connection relationship but also an indirect connection relationship through an intermediate component may also be included. In addition, when a component is referred to as “comprising” or “having” another component, it may mean further inclusion of another component not the exclusion thereof, unless explicitly described to the contrary.

In the present disclosure, the terms first, second, etc. are used only for the purpose of distinguishing one component from another, and do not limit the order or importance of components, etc., unless specifically stated otherwise. Thus, within the scope of this disclosure, a first component in one exemplary embodiment may be referred to as a second component in another embodiment, and similarly a second component in one exemplary embodiment may be referred to as a first component.

In the present disclosure, components that are distinguished from each other are intended to clearly illustrate each feature. However, it does not necessarily mean that the components are separate. That is, a plurality of components may be integrated into one hardware or software unit, or a single component may be distributed into a plurality of hardware or software units. Thus, unless otherwise noted, such integrated or distributed embodiments are also included within the scope of the present disclosure.

In the present disclosure, components described in the various embodiments are not necessarily essential components, and some may be optional components. Accordingly, embodiments consisting of a subset of the components described in one embodiment are also included within the scope of the present disclosure. In addition, exemplary embodiments that include other components in addition to the components described in the various embodiments are also included in the scope of the present disclosure.

Hereinafter, examples of an apparatus and a method for controlling spatially selective air sampling according to one embodiment of the present invention will be described. The accompanying drawings are not necessarily to scale and in some instances, proportions may have been exaggerated in order to clearly illustrate features of the embodiments. Further, the terms to be described below are terms defined in consideration of functions in the present invention and thus may vary according to intentions or customs of users and operators. Accordingly, the definitions of such terms should be made based on the content throughout the specification.

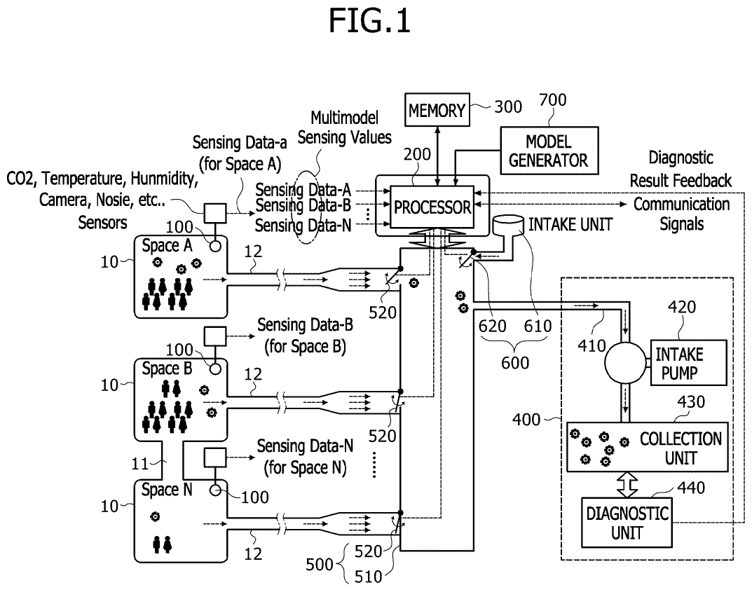

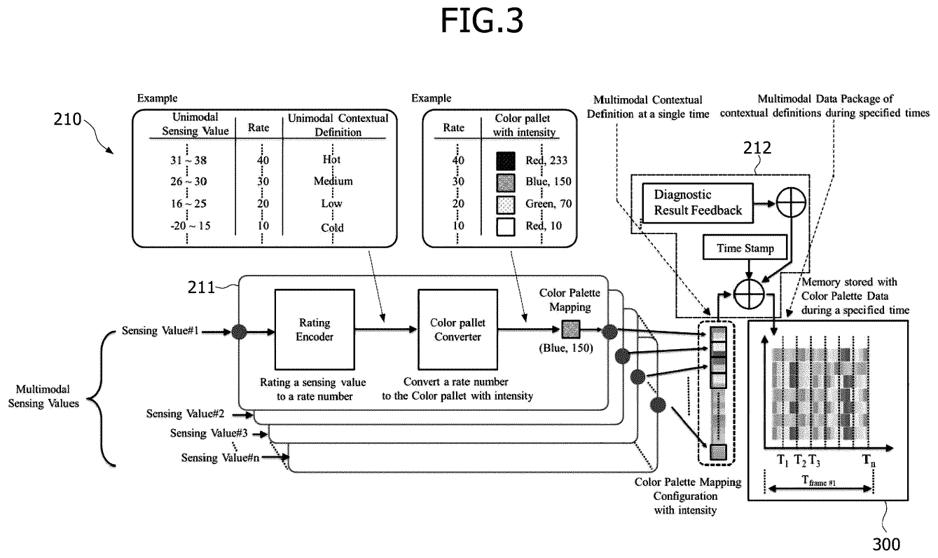

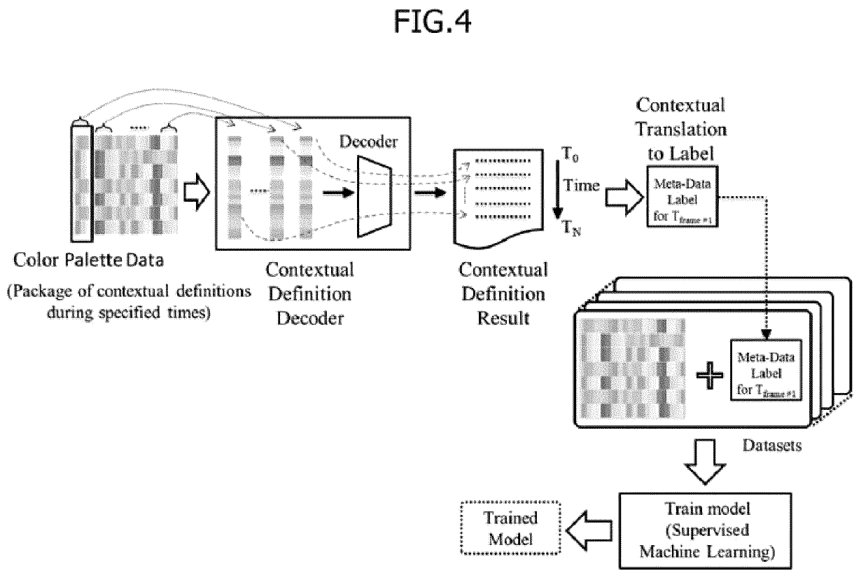

FIG. 1 is a block diagram of an apparatus for controlling spatially selective air sampling according to one embodiment of the present invention. 2 is a block diagram of a processor according to one embodiment of the present invention. FIG. 3 is a block diagram of a color palette generator 210 according to one embodiment of the present invention. FIG. 4 is a diagram illustrating a model generating process of a model generator according to one embodiment of the present invention.

Referring to FIG. 1, the apparatus for controlling spatially selective air sampling according to one embodiment of the present invention may include a sensing unit 100, a processor 200, a memory 300, a diagnostic module 400, an air adjuster 500, a filter module 600, and a model generator 700.

The memory 300 may store various types of data used by the processor 200. Instructions for performing operations, steps, or the like according to one embodiment of the present invention may be stored as data. As an example, the memory 300 generates color palette data by mapping a multimodal sensing value with multimodality characteristics to a unified data space 10 and may generate an instruction for selectively collecting air in each space 10 based on the color palette data.

In addition, the memory 300 may store a diagnostic result for the air in the space 10 and the color palette data.

The memory 300 may include at least one storage medium of a flash memory type, hard disc type, multimedia card micro type, or card type memory, a random access memory (RAM), a static random access memory (SRAM), a read-only memory (ROM), a programmable read-only memory (PROM), an erasable programmable read-only memory (EPROM), and an electrically erasable programmable read-only memory (EEPROM).

At least one sensing unit 100 may be provided in each space 10 to detect the multimodal sensing value with multimodality characteristics.

The sensing unit 100 may input the detected multimodal sensing value to the processor 200.

The sensing unit 100 may be provided as various sensor assemblies that detect the multimodal sensing value.

The sensing unit 100 may detect an interaction between an environment of the space 10 and objects. The environment of the space 10 may include a temperature, humidity, and CO₂ inside the space 10. An interaction between objects may include an image, a voice, and a noise magnitude. The multimodal sensing value detected by the sensing unit 100 is not particularly limited.

The multimodal sensing value may be a value in which pieces of sensing data input from the sensing units 100 are coupled to each other.

The multimodal sensing value may be used to monitor an environment of each space 10 or conditions of objects inside the space 10.

The multimodal sensing value may be image data, voice data, an analog signal, or a digital signal, and the form of the multimodal sensing value is not particularly limited.

The space 10 may be a crowded or closed space 10 and may be a conference room, a religious facility, or a closed animal breeding facility, but is not particularly limited.

In FIG. 1, an example in which space A, space B, and space C are present as the spaces 10, space A is a single space 10, and space B and space C are connected through a connector 11 is shown.

An air tube 12 may be connected to each space 10.

The air tube 12 may suction fine suspended materials such as viruses in air inside the space 10 together with the air and discharge the fine suspended materials to a duct 510 of the air adjuster 500.

The suspended fine material may be suctioned together with the air in each space 10 through the air tube 12 and collected. This will be described below.

The air adjuster 500 may suction air from each space 10 and transfer the suctioned air to the diagnostic module 400.

The air adjuster 500 may filter the fine suspended materials in the air of each space 10.

The air adjuster 500 may include an internal air valve 520 and the duct 510.

The duct 510 may be connected to each space 10 through the air tube 12. The duct 510 may transfer air suctioned from each space 10 to the diagnostic module 400 through the air tube 12.

The internal air valve 520 may suction air in each space 10 through the air tube 12 or block the air according to a control signal of the processor 200.

The internal air valve 520 may be installed in each air tube 12 and individually turned on or off to selectively introduce air from each space 10 to the duct 510.

The filter module 600 may filter air inside the duct 510 of the air adjuster 500 by suctioning outside air.

The filter module 600 may include an intake unit 610 and the internal air valve 520.

The intake unit 610 may be connected to the duct 510 through an outside air pipe.

The intake unit 610 may suction and filter outside air and supply the filtered outside air to the duct 510.

The internal air valve 520 may be installed on the outside air pipe.

The internal air valve 520 may be turned on or off according to a control signal of the processor 200 to allow or block inflow of air from the intake unit 610 to the duct 510.

The diagnostic module 400 may collect fine suspended materials flowing into each space 10 and diagnose a composition of the fine suspended materials.

The diagnostic module 400 may include an intake pipe 410, an intake pump 420, a collection unit 430, and a diagnostic unit 440.

The intake pipe 410 may transfer air inside the duct 510 to the collection unit 430.

Here, fine suspended materials may be present in the air inside the duct 510.

The intake pump 420 may forcibly suction air from the duct 510 through the intake pipe 410 to transfer to the suctioned air to the collection unit 430.

The collection unit 430 may collect fine suspended materials in the air transferred through the intake pipe 410.

The diagnostic unit 440 may diagnose a composition of fine suspended materials in air inside the collection unit 430. The diagnostic unit 440 may transmit a diagnostic result for the composition of the fine suspended materials to the processor 200.

The processor 200 may be connected to the memory 300 and execute instructions stored in the memory 300. The processor 200 may execute the instructions stored in the memory 300, control at least one of other components (for example, a hardware or software component) connected to the processor 200, and perform various data processing or calculations.

In addition, the processor 200 may be provided such that a component for performing each function is divided at a hardware, software, or logic level. In this case, dedicated hardware may be used to perform each function. To this end, the processor 200 may be implemented as an application specific integrated circuit (ASIC), a digital signal processor (DSP), a programmable logic device (PLD), a field programmable gate array (FPGA), a central processing unit (CPU), and/or a microcontroller or include at least one thereof.

The processor 200 may be implemented as a CPU or a system-on-chip (SoC), may drive an operating system or application to control a plurality of hardware or software components connected to the processor 200, and may process various pieces of data and perform calculations. The processor 200 may be configured to execute at least one instruction stored in the memory 300 and store execution result data in the memory 300.

The processor 200 may receive a multimodal sensing value from each sensing unit 100.

The processor 200 may receive a diagnostic result for a composition of fine suspended materials from the diagnostic unit 440.

The processor 200 may generate color palette data using the multimodal sensing value.

The processor 200 may infer a situation for each space 10 according to the color palette data based on a learning model. The processor 200 may collect fine suspended materials in each space 10 by controlling each internal air valve 520 according to an inference result.

The learning model will be described below.

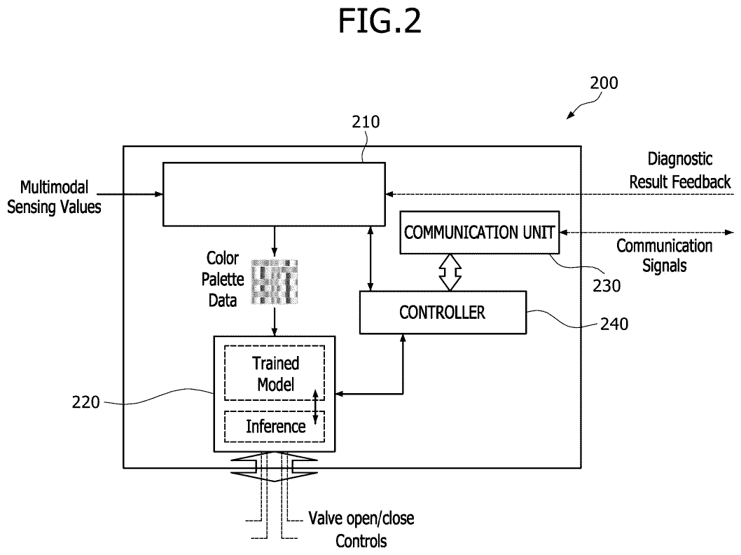

Referring to FIG. 2, the processor 200 may include a color palette generator 210, an inference unit 220, a communication unit 230, and a controller 240.

The color palette generator 210 may use a multimodal sensing value to generate color palette data of a unified data space with pixel brightness corresponding to signal intensity of the multimodal sensing value and may match the generated color palette data with a diagnostic result.

The color palette generator 210 may receive a multimodal sensing value from each sensing unit 100. For example, the color palette generator 210 may receive a bundle of individual unimodal sensing values detected in space A from the sensing unit 100 installed in space A, may receive individual unimodal sensing values detected in space B from the sensing unit 100 installed in space B, and may receive individual unimodal sensing values detected in space C from the sensing unit 100 installed in space C.

Referring to FIG. 3, the color palette generator 210 may include a sensing channel mapper 211 and a data matching unit 212.

The sensing channel mapper 211 may receive a multimodal sensing value from the sensing unit 100.

The sensing channel mapper 211 may encode the multimodal sensing value to define a rate of the multimodal sensing value to a sensing range.

The rate of the multimodal sensing value to the sensing range may be defined as a unimodal contextual definition.

The sensing channel mapper 211 may map a converted sensing range to a color palette with pixel brightness (signal intensity) to corresponding to signal intensity of a detected value (color palette mapping). That is, color data with pixel brightness may be mapped and output inside the sensing channel mapper 211.

In this way, the multimodal sensing value may be mapped to a color with individual brightness by the sensing channel mapper 211.

The data matching unit 212 may periodically generate color palette data according to a preset setting cycle and match a diagnostic result with the color palette data as meta-data.

To this end, the data matching unit 212 may perform a timestamp function to generate a time corresponding to the setting cycle. The data matching unit 212 may match individual color collections (color palette mapping configuration with intensity) with a time corresponding to the setting cycle to generate individual color collections during a corresponding setting sensing. The data matching unit 212 may combine the individual color collections to generate color palette data for a set time section.

The data matching unit 212 may store the above-described individual color collections and color palette data in the memory 300.

Here, the individual color collection is obtained by mapping pieces of individual color data at a specified time and may be consist of a multimodal contextual definition at a corresponding specified time.

The individual color collection may constitute a set time section together with a timestamp over time and may be stored in the memory 300. In this way, data stored in the memory 300, that is, the individual color collection may constitute color palette data. As a result, the color palette data may be defined as a multimodal contextual definition package determined within a set time section.

In addition, the data matching unit 212 may match a diagnostic result with the color palette data as meta-data. Accordingly, a diagnostic result of the diagnostic module 400 may be recorded in the color palette data in the form of meta-data.

The inference unit 220 may infer a situation for each space 10 according to the color palette data based on a learning model. The learning model may be generated by the model generator 700.

The communication unit 230 may perform data communication with an external device.

The controller 240 may control the color palette generator 210 to generate color palette data through a multimodal sensing value and may match the generated color palette data with a diagnostic result.

In addition, the controller 240 controls the inference unit 220 to perform inference according to the color palette data and controls each internal air valve 520 according to an inference result to collect fine suspended materials for each space 10.

Although, in the present embodiment, to facilitate understanding of the embodiment, the color palette generator 210, the inference unit 220, the controller 240, and the communication unit 230 are described as separate components in the processor 200, according to embodiments, the color palette generator 210, the inference unit 220, the controller 240, and the communication unit 230 may be implemented as components in which the processor 200 integrally performs each sub-configuration.

The model generator 700 may generate an inference model.

Referring to FIG. 4, an individual color collection may refer to a multimodal contextual definition at a specified time, and color palette data may refer to a contextual definition package during a set time section.

In this case, the model generator 700 may decode the individual color collection to obtain an individual situation for each specified time and may generate a contextual definition result according to time information with the individual situation during the specified time. In this case, a decoding method may be defined according to the type and characteristics of a detected value.

The model generator 700 may convert the contextual definition result into a meta-data label according to contextual translation of a user. The contextual translation corresponding to the time section is expressed as the meta-data label.

The model generator 700 may match the converted meta-data label with color palette data and construct a dataset of meta-data label and color palette data as a pair.

The model generator 700 may train a model using the dataset, that is, the color palette data and the meta-data.

The model generator 700 may transfer the generated model to the processor 200 to allow the model to be mounted on the processor 200.

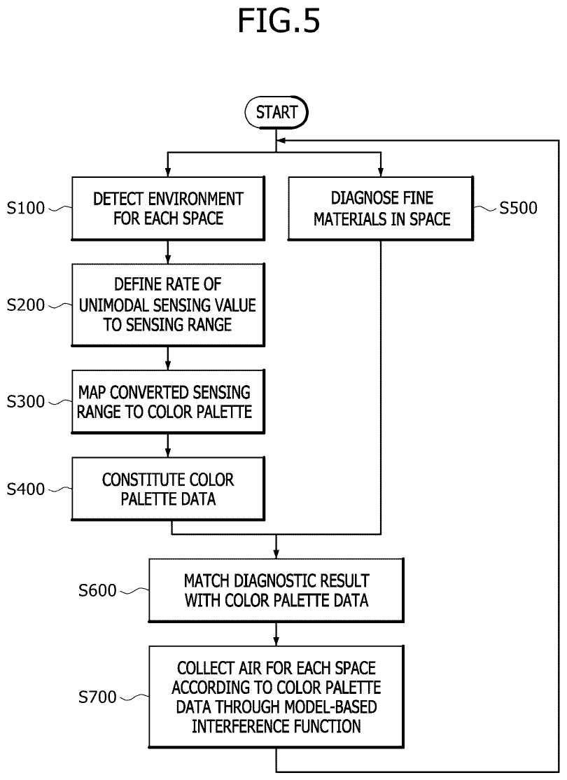

Hereinafter, a method of controlling spatially selective air sampling according to one embodiment of the present invention will be described with reference to FIG. 5.

FIG. 5 is a flowchart of the method of controlling spatially selective air sampling according to one embodiment of the present invention.

Referring to FIG. 5, a plurality of sensing units 100 may be provided in each space 10 and detect a multimodal sensing value having multimodality characteristics (S100).

In this case, the sensing unit 100 may detect an interaction between an environment of the space 10 and objects, and the environment of the space 10 may include a temperature, humidity, and CO₂ inside the space 10, and an interaction between the objects may include an image, a voice, and a noise magnitude.

The processor 200 may receive the multimodal sensing value from each sensing unit 100. The processor 200 and the sensing channel mapper 211 may define a rate of the multimodal sensing value to a sensing range (S200). The rate of the multimodal sensing value to the sensing range may be defined as a unimodal contextual definition.

The processor 200 may map a converted sensing range to a color palette with pixel brightness (signal intensity) (S300). That is, a color with pixel brightness may be mapped and output inside the sensing channel mapper 211.

The processor 200 may perform a timestamp function to generate a time corresponding to a setting cycle and match individual color configurations to the time corresponding to the setting cycle to generate individual color configurations for a set time section. The processor 200 may generate color palette data for the set time section by combining the individual color collections (S400). In this case, the data matching unit 212 may store the above-described individual color collection and color palette data in the memory 300.

Meanwhile, the diagnostic module 400 may collect fine suspended materials flowing into each space 10 and diagnose a composition of fine suspended materials (S500).

Accordingly, the processor 200 may match a diagnostic result with the color palette data as meta-data (S600).

Next, the processor 200 may infer a situation for each space 10 according to the color palette data based on a learning model. The processor 200 controls each valve 520 according to an inference result to collect fine suspended materials in each space 10 and allows the fine suspended materials to be diagnosed through the diagnostic module 400 (S700).

According to an apparatus and a method for controlling spatially selective air mapping according to one aspect of the present invention, multimodal sensing values with multimodality characteristics such as image data, voice data, an analog signal, and a digital signal can be mapped to one unified data space.

According to an apparatus and a method for controlling spatially selective air mapping according to another aspect of the present invention, even when the number of sensors considerably increases, input signals thereof are mapped to one unified data space, thereby facilitating contextual translation.

According to an apparatus and a method for controlling spatially selective air mapping according to still another aspect of the present invention, air tubes are individually connected to a plurality of crowded spaces to allow one device to selectively suction air from each crowded space or collect and diagnose fine suspended materials in the air, thereby improving economic feasibility of improving air quality in the crowded spaces.

The present invention has been described with reference to embodiments shown in the accompanying drawings, but this is merely illustrative, and those skilled in the art will understand that various modifications and other equivalent embodiments are possible therefrom. Therefore, the technical protection scope of the present invention should be defined by the appended claims.

What is claimed is:

1. An apparatus for controlling spatially selective air sampling, the apparatus comprising:
a sensing unit configured to detect a multimodal sensing value for at least one space;
a diagnostic module configured to collect fine suspended materials in air of the space and diagnose a composition of the fine suspended materials;
an air adjuster configured to suction the air of the space and transfer the suctioned air to the diagnostic module; and
a processor configured to generate color palette data of a unified data space having pixel brightness corresponding to signal intensity of the multimodal sensing value, map the color palette data to a diagnostic result of the diagnostic module, perform inference through a learning model based on the color palette data and the diagnostic result, and control the air adjuster for each space according to an inference result to transfer the air to the diagnostic module.

2. The apparatus of claim 1, wherein the diagnostic module includes:
a collection unit configured to collect the fine suspended materials in the air of the air adjuster; and
a diagnostic unit configured to diagnose the composition of the fine suspended materials in the collection unit.

3. The apparatus of claim 1, wherein the air adjuster includes:
a duct connected to the space to transfer the air suctioned from the space through an air tube to the diagnostic module; and
an internal air valve installed on the air tube to block the air in the space and introduce the air into the duct.

4. The apparatus of claim 3, further comprising a filter module configured to suction and filter outside air and supply the filtered outside air to the duct.

5. The apparatus of claim 1, wherein the processor defines a rate of a sensing range of the multimodal sensing value, maps the sensing range to a color palette, and generates individual color collections for each preset setting cycle.

6. The apparatus of claim 5, wherein the processor generates the color palette data by combining the individual color collections in a preset time section.

7. The apparatus of claim 5, further comprising a model generator configured to decode the individual color collection to obtain an individual situation, generate a contextual definition result according to time information with the individual situation, convert the contextual definition result into a meta-data label according to contextual translation of a user, match the meta-data label with the color palette data to constitute a dataset, and then train the learning model using the dataset.

8. An apparatus for controlling spatially selective air sampling, the apparatus comprising:
a processor; and
a memory configured to store an instruction executed by the processor,
wherein the processor uses a multimodal sensing value for at least one space to generate color palette data of a unified data space having pixel brightness corresponding to signal intensity of the multimodal sensing value.

9. The apparatus of claim 8, wherein the processor defines a rate of the multimodal sensing value to a sensing range, maps the sensing range to a color palette, and generates individual color collections for each preset setting cycle.

10. The apparatus of claim 9, wherein the processor generates the color palette data by combining the individual color collections in a preset time section.

11. The apparatus of claim 9, wherein the processor decodes the individual color collection to obtain an individual situation, generates a contextual definition result according to time information with the individual situation, converts the contextual definition result into a meta-data label according to contextual translation of a user, matches the meta-data label with the color palette data to constitute a dataset, and then train a learning model using the dataset.

12. A method of controlling spatially selective air sampling, the method comprising:
detecting, by a sensing unit, a multimodal sensing value for at least one space;
collecting, by a diagnostic module, fine suspended materials in air of the space and diagnosing a composition of the fine suspended materials;
generating, by a processor, color palette data in a unified data space having pixel brightness corresponding to signal intensity of the multimodal sensing value using the multimodal sensing value;
mapping, by the processor, the color palette data to a diagnostic result of the diagnosis module; and
performing, by a processor, inference, based on the color palette data and the diagnostic result using a learning model, controlling an air adjuster for each space according to an inference result, and transferring the air to the diagnosis module.

13. The method of claim 12, wherein, in the generating of the color palette data, the processor defines a rate of a sensing range of the multimodal sensing value, maps the sensing range to a color palette, and generates an individual color collection for each preset setting cycle.

14. The method of claim 13, wherein, in the generating of the color palette data, the processor generates the color palette data by combining the individual color collections in a preset time section.

15. The method of claim 13, wherein the learning model decodes the individual color collection to obtain an individual situation, generates a contextual definition result according to time information with the individual situation, converts the contextual definition result into a meta-data label according to a contextual translation of a user, matches the meta-data label with the color palette data to constitute a dataset, and then is trained using the dataset.
